(19) United States
(12) Patent Application Publication
(10) Pub. No.: US 2013/0121922 A1
(43) Pub. Date: May 16, 2013

(54) METHODS FOR EVALUATING AND SELECTING DERMAL FILLER COMPOSITIONS

(71) Applicant: ALLERGAN, INC., Irvine, CA (US)

(72) Inventors: Sumit Paliwal, Goleta, CA (US); Dennis Van Epps, Goleta, CA (US)

(73) Assignee: ALLERGAN, INC., Irvine, CA (US)

(21) Appl. No.: 13/691,196

(22) Filed: Nov. 30, 2012

Related U.S. Application Data
(63) Continuation-in-part of application No. 13/672,490, filed on Nov. 8, 2012.
(60) Provisional application No. 61/565,832, filed on Dec. 1, 2011.
(60) Provisional application No. 61/558,325, filed on Nov. 10, 2011.

(51) Int. Cl. A61K 49/00

(52) U.S. Cl. CPC A61K 49/0006
USPC 424/9.2

(57) ABSTRACT

The present invention provides methods for selecting a cosmetically acceptable and effective dermal filler, with reduced chance of causing discoloration in skin of a patient.

This application claims priority to U.S. Provisional Patent Application No. 61/565,832, filed Dec. 1, 2011, and is a continuation-in-part of U.S. patent application Ser. No. 13/672,490, filed Nov. 8, 2012, which claims priority to U.S. Provisional Patent Application No. 61/558,325, filed Nov. 10, 2011, the entire contents of which are incorporated herein by reference.

The present disclosure generally relates to methods for evaluating and selecting aesthetic dermal fillers, and more specifically relates to methods for evaluating discoloration in skin for example, as a result of Tyndall effect, of such fillers.

### BACKGROUND

Skin aging is a progressive phenomenon, occurs over time and can be affected by lifestyle factors, such as alcohol consumption, tobacco and sun exposure. Aging of the facial skin can be characterized by atrophy, slackening, and fattening. Atrophy corresponds to a massive reduction of the thickness of skin tissue. Slackening of the subcutaneous tissues leads to an excess of skin and ptosis and leads to the appearance of drooping cheeks and eye lids. Fattening refers to an increase in excess weight by swelling of the bottom of the face and neck.

Hyaluronic acid (HA), also known as hyaluronan, is a glycosaminoglycan that is distributed widely throughout the human body in connective, epithelial, and neural tissues. Hyaluronic acid is abundant in the different layers of the skin, where it has multiple functions such as, e.g., to ensure good hydration, to assist in the organization of the extracellular matrix, to act as a filler material; and to participate in tissue repair mechanisms. However, with age, the quantity of hyaluronic acid, collagen, elastin, and other matrix polymers present in the skin decreases. For example, repeated exposed to ultra violet light, e.g., from the sun, causes dermal cells to both decrease their production of hyaluronan as well as increase the rate of its degradation. This loss of materials results in various skin conditions such as, e.g., wrinkling, hollowness, loss of moisture and other undesirable conditions that contribute to the appearance of aging.

Injectable dermal fillers have been successfully used in treating the aging skin. The fillers can replace lost endogenous matrix polymers, or enhance/facilitate the function of existing matrix polymers, in order to treat these skin conditions. Hyaluronic acid-based dermal fillers have become increasingly popular, as hyaluronic acid is a substance naturally found throughout the human body. These fillers are generally well tolerated, nonpermanent, and a fairly low risk treatment for a wide variety of skin conditions.

Tyndall effect is an adverse event occurring in some patients administered with hyaluronic acid (HA)-based dermal fillers. Tyndall effect is characterized by the appearance of a blue discoloration at the skin site where a dermal filler had been injected, which represents visible hyaluronic acid seen through the translucent epidermis. Clinical reports suggest that filler administration technique and skin properties can influence the manifestation of this adverse event. Fillers with high stiffness and elasticity are successfully used to correct areas on the face like nasolabial folds, cheeks, and chin without any fear of facial discoloration, as the materials are injected in the mid and deep dermis regions. However, when these filler materials are used to correct superficial, fine line wrinkles, for example, tear trough, glabellar lines periorbital lines, smile lines, or forehead, or mistakenly applied too superficially in the upper regions of the dermis, a bluish discoloration of the skin is often observed. This phenomenon, which is thought to be the result of Tyndall effect, leaves a semi-permanent discoloration of the application sites, and sometimes disappears only after the administration of hyaluronidase to degrade the filler material. Consequently, Tyndall effect is more common in patients treated for superficial fine line wrinkles. Prolonged manifestation of Tyndall effect, typically for several months as long as the gel lasts in the skin, is a cause of major concern among patients.

Commercial dermal filler gels have been specifically formulated to treat “fine line” wrinkles found around the tear trough, forehead, periorbital, glabellar lines, etc. Many of these commercially available fine line gels show discoloration of skin, believed to be a result of Tyndall effect, particularly when injected too superficially.

Prolonged manifestation of Tyndall effect until the gel lasts in the skin (typically for several months) is a cause of major concern among patients. Despite these concerns, it is unfortunate that there are no current methods available for a priori evaluation of HA fillers to predict whether a specific filler will manifest Tyndall effect. Furthermore, no methods exist to quantify Tyndall effect and determine the performance of HA fillers. Shortage of such methods has significantly impeded the discovery process of novel fillers that will not show Tyndall effect.

Notably, the degree or severity of Tyndall effect may vary between patients. Thus, what may cause severe Tyndall effect in one patient may cause little or no perceptible Tyndall effect in another patient.

The present disclosure provide methods for evaluating dermal filler compositions, for example, selecting dermal fillers which will have lowest chance of manifesting Tyndall effect for a patient or patient population.

### SUMMARY

The present disclosure provides methods for evaluating dermal filler compositions, for example, for predicting manifestation of and/or quantifying, discoloration due to Tyndall effect. In addition, methods are provided for selecting an effective, cosmetically acceptable dermal filler composition for use in a patient, for example, from a selection of different dermal filler compositions, wherein the selected dermal filler composition will successfully treat fine line wrinkles while also manifesting no perceptible Tyndall effect or other discoloration.

In one aspect, the present disclosure provides useful methods for identifying HA-based dermal fillers which will be cosmetically acceptable and will enhance the appearance of the skin without causing Tyndall effect when implanted superficially and/or into thin skin, such as the periocular region, the periorbital region or tear trough.

Generally, the methods comprise the steps of quantifying a color component in a skin region of a patient and selecting a dermal filler composition, for example, from a selection of different dermal filler compositions, based on the quantified color component of the skin region of the patient.

In one embodiment, the step of quantifying comprises introducing into a skin region a dermal filler composition to be evaluated, and observing the skin region for manifestation of discoloration, for example, blue discoloration. If undesirable discoloration is observed, for example, blue discoloration as a result of Tyndall, the dermal filler can be eliminated from the selection. Another dermal filler may be then evaluated, for example, in a similar manner, for suitability thereof for the patient.

In some embodiments, the step of observing comprises comparing a color component observed in the treated skin region with the same color component observed in an untreated skin region. In addition, the skin region and the untreated skin region may be compared to one another. For example, the color component (e.g. blue) of the treated skin region may be compared with the same color component (in this case, also e.g. blue) of the untreated skin region of the same patient. The skin region having the composition introduced therein, hereinafter sometimes referred to as the “treated skin” or “treated skin region”, may be compared to a untreated skin region (“untreated skin” or “untreated skin region”) for example, of the same patient.

In one embodiment, the step of observing comprises measuring at least one color component of the treated skin region. The color component measured may include a blue color component, which may indicate Tyndall effect.

In one embodiment, the step of quantifying further comprising reducing blood flow to the skin region prior to or during the step of observing. This may be accomplished by any suitable means, for example, introducing a vasoconstrictor to the skin region, applying a vasoconstricting agent to the skin region, for example, topically or intradermally. The step of reducing blood flow may include cooling the skin region, for example, by applying ice or a cold compress to the skin region.

In one aspect, the blood flow is reduced within a certain defined time period following the introduction of the composition. For example, in some embodiments, the vasoconstrictor is introduced to the skin region no sooner than between about 12 hours and about 60 hours after the step of introducing the composition.

Measuring of the color component, in some embodiments, is accomplished by assessing the discoloration using visual observations and comparisons. Alternatively or additionally, the measuring may be accomplished using an electromechanical device, for example, using instruments capable of measuring color and components of color using reflectance spectroscopy. For example, a percentage (%) of blue light reflected from the skin region may be quantified using a suitable instrument, such as a spectrophotometer. Measurements may be made at spaced apart locations in the skin region, and the measurements averaged. Based on the measurements, a dermal filler can be selected which will have a low likelihood of exhibiting Tyndall effect (or other discoloration) when injected into the skin to treat, for example, superficial wrinkles (fine lines) of the face of the patient.

In another aspect, measurements of one or more color components of the treated skin region may be compared with such color components of untreated skin, and such comparison may be used to quantify a degree or amount of discoloration, if any, resulting from the presence of the composition in the treated skin region. A dermal filler composition may then be eliminated or selected as being suitable to treat the patient.

Measuring may include quantifying a blue color component of the skin region and assigning the discoloration a grade based on a numerical scale. In one embodiment, the quantifying comprises visually observing the skin region and assigning to the observed skin region a grade on a scale of 1 to 5. Quantifying a color component may include quantifying the blue color component on a I-a-b color scale, alternatively or additionally, quantifying a color component may include quantifying a percentage (%) of blue light remitted from the skin region using a suitable electromechanical instrument, for example, an spectrophotometer. Measuring may further include quantifying a color component, for example, a blue color component of the skin region, and comparing the blue color component of treated skin region with a blue color component of untreated skin.

Aspects of this disclosure further include methods for evaluating a hyaluronic acid-based dermal filler for its potential for causing discoloration in skin when introduced into skin. Other aspects of this disclosure include methods for determining potential for discoloration of skin caused by a dermal filler composition being introduced therein. In some embodiments, these methods comprise introducing, for example, using linear threading technique, into a skin region of a patient, a composition to be evaluated, reducing blood flow to the skin region after the step of introducing the composition, measuring discoloration in the skin region using an electromechanical device, and comparing the measured discoloration with an area of untreated skin of the patient.

Although the present disclosure describes primarily, methods for selecting dermal fillers for a patient which will not manifest perceptible blue discoloration due to Tyndall effect, it is to be appreciated that the presently described methods may be used for selecting dermal fillers for a patient which will not manifest other perceptible discoloration based on non-blue or other color components of the skin in addition to, or alternative to, a blue color component.

### BRIEF DESCRIPTION OF THE DRAWINGS

These and other aspects of this disclosure may be more readily understood and appreciated with reference to the following detailed description and accompanying drawings of which:

FIG. 1 shows images of skin of test animals after superficial injection of different optically transparent, HA-based dermal filler compositions, in order to show how different optically transparent dermal fillers will sometimes manifest Tyndall effect when injected into skin;

FIG. 2 shows steps of a linear injection technique useful in methods for evaluating dermal filler compositions;

FIG. 3 is a diagram of locations of spaced apart measurement in a treated and untreated skin regions.

FIG. 4 shows a bar graph representing visual discoloration scores of skin regions injected with different dermal filler compositions;

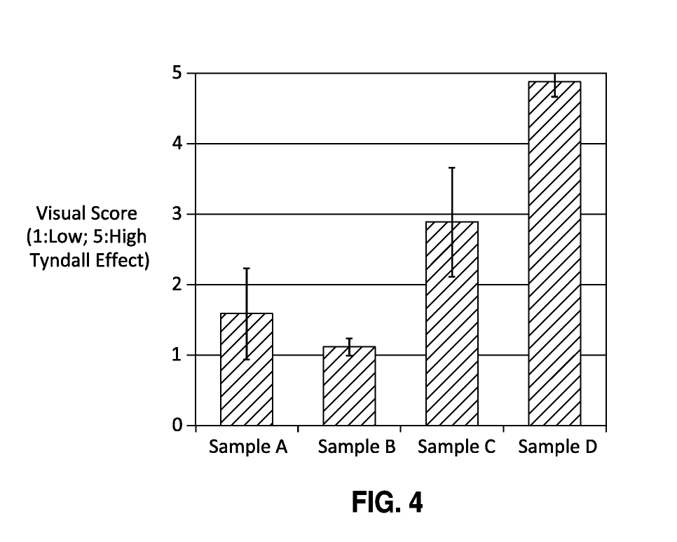

FIG. 5 shows a bar graph representing blue color component (as defined by l-a-b color system) of skin regions injected with different HA-based dermal filler compositions; and

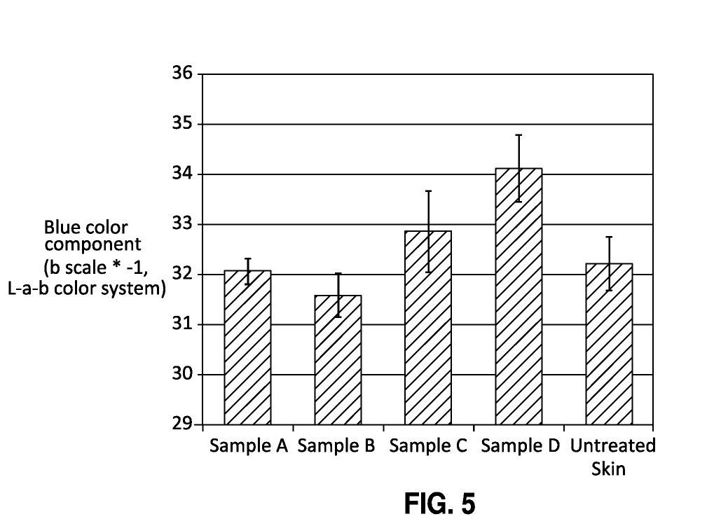

FIG. 6 shows a bar graph representing percentage (%) of blue light reflected from skin injected with different HA-based dermal filler compositions.

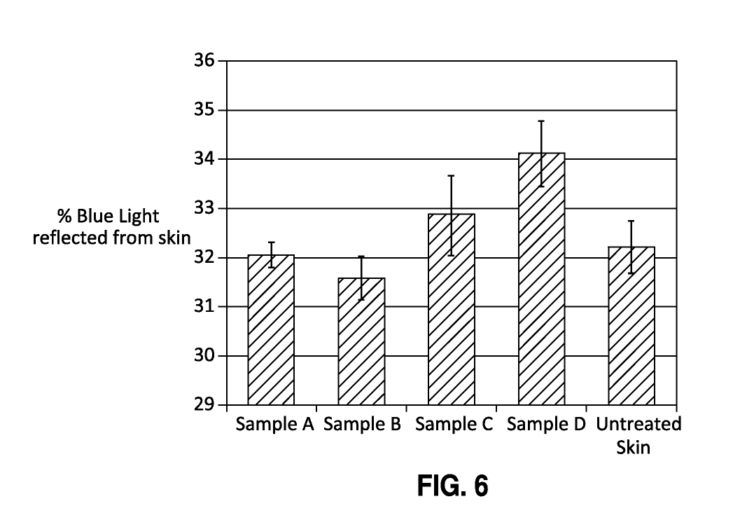

### DETAILED DESCRIPTION

Generally, methods for evaluating dermal filler compositions for the potential to cause discoloration, for example, Tyndall effect, in skin, are provided. The compositions which may be tested with these methods include any compositions which could potentially cause discoloration when introduced into skin, for example, human skin. Such discoloration may not be due to a physiological reaction of the body to the composition, but is sometimes a result of selective reflectance or absorbance of visible light of the composition through the skin, for example what is commonly known as “Tyndall effect”. Tyndall effect commonly manifests as a blue discoloration in thin or fair skinned individuals (for example, but not limited to individuals having Fitzpatrick skin type II and III) when certain compositions are injected too superficially.

Unfortunately, Tyndall effect persists in the skin until the composition is removed from the skin, for example, by biodegradation or otherwise. Compositions exhibiting Tyndall effect include, but are not limited to, optically transparent, substantially optically transparent, biocompatible polymers, for example, polysaccharides such as crosslinked hyaluronic acid (HA)-based compositions.

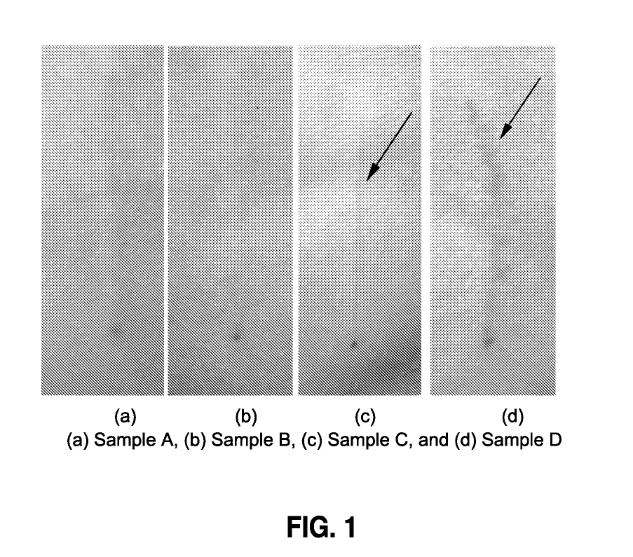

In order to illustrate manifestation of Tyndall effect in skin, FIG. 1 is provided which shows discoloration of skin regions of four animals, in this case, hairless rats, as a result of different dermal filler compositions introduced superficially therein. Skin regions in photos c and d show marked discoloration, specifically blue discoloration, as a result of Tyndall effect. Tyndall effect, as mentioned elsewhere herein, is a significant adverse event experienced by some dermal filler patients. Prediction of which dermal fillers will not exhibit Tyndall effect in fair skinned individuals has been problematic. It is not readily apparent from the physical or chemical properties of these compositions, nor from the skin types of the individuals, which compositions will exhibit Tyndall effect, and which ones will not.

The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells and Merkels cells. The dermis is the layer of skin beneath the epidermis that consists of connective tissue and cushions the body from stress and strain. The dermis is tightly connected to the epidermis by a basement membrane. It also harbors many mechanoreceptor/nerve endings that provide the sense of touch and heat. It contains the hair follicles, sweat glands, sebaceous glands, apocrine glands, lymphatic vessels and blood vessels. The blood vessels in the dermis provide nourishment and waste removal from its own cells as well as from the Stratum basale of the epidermis. The dermis is structurally divided into two areas: a superficial area adjacent to the epidermis, called the papillary region, and a deep thicker area known as the reticular region.

Fine lines or superficial wrinkles are generally understood to be those wrinkles or creases in skin that are typically found in regions of the face (forehead, lateral canthus, vermillion border/perioral lines) where the skin is thinnest, that is, the skin has a dermis thickness of less than 1 mm. On the forehead the average dermal thickness is about 0.95 mm for normal skin and about 0.81 mm for wrinkled skin. Dermis around the lateral canthus is even thinner (e.g. about 0.61 mm for normal skin and about 0.41 mm for wrinkled skin). The average outer diameter of a 30 or 32 gauge needle (needles that are typically used for fine line gel application) is about 0.30 and about 0.24 mm.

Tyndall effect often manifests in patients treated for superficial fine line wrinkles. Thus, aspects of this disclosure provide methods for evaluating dermal fillers for potential to cause Tyndall effect in a patient. Methods for evaluating compositions and identifying those which will not exhibit significant discoloration, for example, from Tyndall effect, when the compositions are used to treat fine lines, are provided.

Compositions with reduced risk of discoloration when introduced into skin may be identified in accordance with the present disclosure, wherein such compositions are dermal fillers useful for treating various skin conditions. Compositions may be identified which are useful for fine line treatment of skin, for example, for reducing the appearance of superficial, relative shallow wrinkles in skin, for example, thin skin, of a human being. Compositions may be identified which are useful for treating skin dehydration, wherein such composition, when introduced into the skin, rehydrates the skin, thereby treating skin dehydration, without undesirably discoloring the appearance of the skin. Compositions may be identified which are useful for treating lack of, or reduced, skin elasticity, wherein such composition, when introduced into the skin, increases the elasticity of the skin, without undesirably discoloring the appearance of the skin. Compositions may be identified which are useful for treating skin roughness wherein such composition, when introduced into the skin, decreases skin roughness, without undesirably discoloring the appearance of the skin. Similarly, compositions may be identified which are useful for treating lack of or reduced skin tautness, wherein such composition, when introduced into the skin, makes the skin tauter, without undesirably discoloring the appearance of the skin.

Accordingly, the present disclosure provides methods for evaluating compositions for their potential to cause discoloration before the compositions are used in a therapeutic or cosmetic setting in a patient.

In one embodiment, a method for evaluating a dermal filler composition comprises introducing into a skin region of a patient, a composition to be evaluated, and measuring discoloration of the skin region.

As used herein, the terms “skin region” and “dermal region” refers to the region of skin comprising the epidermal-dermal junction and the dermis including the superficial dermis (papillary region) and the deep dermis (reticular region).

The method may further comprise the step of reducing blood flow to the skin region. Although discoloration from Tyndall effect may manifest sometimes immediately after introduction of the composition into the skin region, the blue discoloration becomes more pronounced over the next couple of days.

In some embodiments, the assessment is made substantially immediately after the introduction of the composition into the skin region. In other embodiments, the discoloration is observed and/or measured after a selected time period has passed to enable more pronounced manifestation of the discoloration. The time period may be, for example, after at least twelve hours, at least twenty four hours, at least thirty six hours, at least forty eight hours, or longer after introduction of the composition into the skin region.

In some embodiments, blood flow is reduced to the skin region, for example, before or after the introduction of the composition to the skin region. In some embodiments the step of reducing blood flow is performed after the selected time period.

Reducing blood flow may be accomplished by any suitable means, for example, by applying (e.g. topically) or introducing (e.g. intradermally) a suitable vasoconstrictor, or vasoconstriction agent to the skin region. Reducing blood flow may further or alternatively be accomplished by reducing the temperature of the skin region, for example, by application of a cold compress or ice to the region. Alternatively, the physician applies a combination of a local anesthetic agent and vasoconstrictor to the treated area in order to both reduce pain of injection and constrict the blood vessels. Some commercially available combinations include, for example, Prilocaine/epinephrine, lidocaine/epinephrine; articaine/epinephrine.

The step of measuring the discoloration may comprise visually assessing the discoloration, for example, visually observing the discoloration by comparing the treated skin region with an untreated skin region. The discoloration may be assigned a grade based on a numerical scale, for example, a scale of 1 to 5 in increments of 0.5. A score of 1 may be assigned to treated skin regions with normal skin tone and no blue discoloration, such as shown in FIG. 1, photo (b). A maximum score of 5 may be assigned to thick and pronounced blue discoloration.

In another aspect, the measuring comprises assessing the discoloration using an electromechanical device. For example, the measuring comprises assessing the discoloration using instruments used to measure color using reflectance spectroscopy, such as a spectrophotometer. A color component of the treated skin region may be quantified in any suitable manner. In some embodiments, the method comprises quantifying a percentage (%) of blue light remitted from the skin region.

In an aspect of this disclosure, the color component measured, for example, the blue color component, may be quantified based on the L-a-b color scale. Unlike other color models, the L-a-b color scale is designed to approximate human vision. Its “L” component closely matches human perception of lightness. Instruments useful for accurately measuring color are available, including instruments referred to as spectrophotometer or chromameter.

To increase accuracy of the assay, the color component may be measured or quantified as described, at a plurality of separate spaced apart locations in the skin region and the measurements averaged. Further, the measurements may be compared to measurements of color of untreated skin.

### EXAMPLE 1

### Method of Evaluating Potential for Discoloration

The following example describes a method for assessing skin discoloration after dermal filler administration, such as discoloration due to Tyndall effect, in a patient.

Prepare filler injection apparatus. In this example, the injection apparatus is a syringe pump connected to a 1″ long 27G needle using a 13″ long sterile silicone cannula. Syringe pump is calibrated to inject filler at a rate of 100 μL/min from the syringe with internal diameter of 4.6 mm.

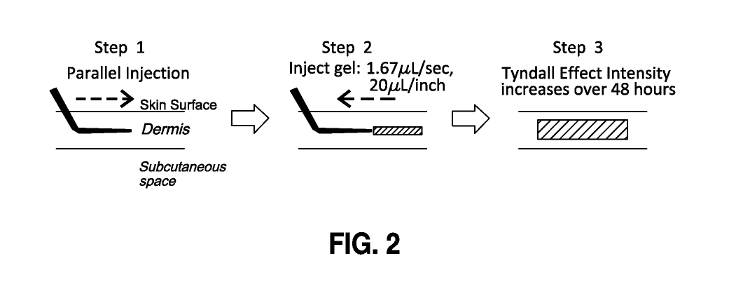

The injection site is numbed with lidocaine if desired. Filler injections are performed using a linear threading technique (see FIG. 2). Samples are placed intradermally in the underside of the forearm. Once inside the dermis, the needle is fully inserted parallel to skin surface. The Tyndall effect starts to develop over time at the filler injection site.

The injections can be (optionally) performed at two locations on the arm. If performing 2 injections.

Ice is applied to the skin region to constrict the blood vessels and enhance the appearance of Tyndall effect, if any.

Discoloration intensity for the treated skin region site can be quantified visually and/or using spectroscopy:

Method 1. Visual Measurement

A Tyndall Effect Visual Score is defined by visually assessing the blue discoloration at the injection site compared to the adjacent untreated skin. The scale has a range of 1 to 5 with increments of 0.5. A score of 1 is given to injection sites with normal skin tone and no blue discoloration. A maximum score of 5 is given to thick and pronounced blue discoloration (typically associated with certain commercial HA based fillers).

Method 2. Spectroscopy Measurement

Reflectance spectroscopy is used to quantitatively assess the blue color of skin. Using this technique two distinct parameters can be defined that independently measure the intensity of blue color in skin, or Tyndall effect. These parameters are described below:

Method 2.1. Blue component of skin color—“b”: A chromameter is used to quantify the blue color component of light remitted from skin sites injected with the various fillers. This is achieved by using the “b” component of L-a-b color scale. The L-a-b color scale uses 3 component (L, a and b) notation that can be used to define any color and is designed to approximate human vision. L defines lightness, and a and b define color-opponent dimensions. Specifically, component b defines color varying from yellow (positive axis) to blue (negative axis). A highly negative “b” component for skin color will mean skin has a strong blue discoloration, as seen in Tyndall effect. L-a-b color aspires to perceptual uniformity, and its L component closely matches human perception of lightness. It can thus be used to make accurate color balance corrections by modifying output curves in the a and b components, or to adjust the lightness contrast using the L component.

Method 2.2. “% Blue Light” remitted from skin: A portable spectrophotometer is be used to quantify the % blue light reflected from skin in the total visible light range.

Spectrophotometer measures the visible light reflected from skin surface, specifically between 400-700 nm wavelengths. The % blue light reflected from skin can be quantified by integrating the area under the visible light spectrum between 400-490 nm and normalizing it by the total area under the spectrum (400-700 nm). An increase in the % blue light as determined from the reflected light spectrum will mean skin has a strong blue discoloration, as seen in Tyndall effect.

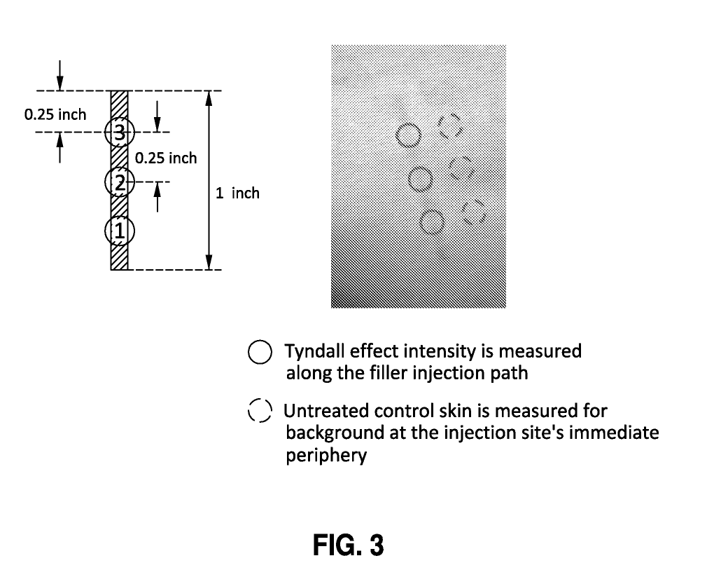

Based on spectroscopic quantification of discoloration, e.g. due to Tyndall effect, each injection site is measured at three equidistant locations along the injection path (see FIG. 3). For a 1″ injection path, each of the measured locations is 0.25″ apart such that the first measured location is positioned 0.25″ from the injection entry location. Finally, the two Tyndall effect intensity parameters ((a) blue component of skin color—“b”, and (b) “% Blue Light” remitted from skin) are calculated for each location. The measurements are further averaged for the three locations along the injection path to calculate a single measurement for the injected skin site. Similar measurements are performed on untreated skin adjacent to the injection site to measure background (untreated skin) discoloration.

By using the method described herein, the intensity of discoloration, for example, from Tyndall effect, and the potential for manifestation of Tyndall effect, in human skin, for example can be evaluated for a particular filler composition.

### EXAMPLE 2

### Visual Assessment and Quantification of Skin Discoloration

FIG. 1 shows different levels of visually apparent discoloration in rats after superficial injection of four different HA-based compositions: Sample A, Sample B, Sample C and Sample D. The samples differ from one another based on, for example, concentration of hyaluronic acid, the inclusion or absence of additives, degree of crosslinking, etc. In this example, each of the samples is substantially entirely clear or optically transparent prior to being placed in the skin. However, as can be appreciated from viewing FIG. 1, the samples manifest blue discoloration, presumably as a result of Tyndall effect, to varying degrees.

The blue discoloration is measured visually and spectroscopically as described elsewhere herein.

### EXAMPLE 3

### Visual Assessment and Quantification of Discoloration in Cadaver Skin

Samples of different HA-based fillers are implanted superficially in freshly excised cadaver skin. Visual and spectroscopic analysis is performed such as described in Example 1. Some of the samples manifest blue discoloration, presumably as a result of Tyndall effect. Other of the samples do not manifest blue discoloration.

The blue discoloration is measured visually and spectroscopically as described elsewhere herein.

### EXAMPLE 4

### Visual Assessment and Quantification of Discoloration in Avian Skin

Samples of different HA-based fillers are implanted superficially in previously frozen chicken skin. Visual and spectroscopic analysis is performed such as described in Example 1. Some of the samples manifest blue discoloration, presumably as a result of Tyndall effect. Other of the samples do not manifest blue discoloration.

The blue discoloration is measured visually and spectroscopically as described elsewhere herein.

### EXAMPLE 5

### Selecting a Suitable Dermal Filler for a Patient from a Selection of Dermal Fillers

A patient desires to have dermal filler treatment for fine lines in the periorbital region, but is concerned about the potential for Tyndall effect. The patient's skin is Fitzpatrick type IV.

A physician suggests evaluation of several commercial dermal fillers from a selection thereof, to determine which filler will successfully reduce or eliminate the appearance of fine line wrinkles without causing undesirable discoloration due to Tyndall effect.

The physician introduces a first hyaluronic-acid based dermal filler intradermally into the right forearm of the patient using linear threading technique. The physician applies and ice pack to the treated skin area for about five minutes to numb the area and effect vasoconstriction.

The physician evaluates the appearance of the skin region and compares it to untreated skin, using a portable spectrophotometer to quantify the % blue light reflected from the skin. The spectrophotometer indicates a reading of 34.2% for the treated skin region and 33.0% for the untreated skin region.

The physician selects a second hyaluronic-acid based dermal filler from the selection. The physician introduces the second intradermally into the right forearm of the patient using linear threading technique, the second dermal filler being introduced about 2 cm apart from the site of the first dermal filler. An ice pack is applied to the injection area.

The physician evaluates the appearance of the second dermal filler treated skin region and compares it to both the first dermal filler treated skin region and the region of untreated skin, using the portable spectrophotometer to quantify the % blue light reflected from the skin. The spectrophotometer indicates a reading of 33.1% for the treated skin region and 33.0% for the untreated skin region.

Based on the measurements, the physician determines that the second dermal filler is the best selection for treating the fine line wrinkles in the patient.

### EXAMPLE 6

### Selecting a Suitable Dermal Filler for a Patient from a Selection of Dermal Fillers

A patient desires to have dermal filler treatment for fine lines in the tear trough region, but is concerned about the potential for Tyndall effect. The patient's skin is Fitzpatrick type II.

A physician suggests evaluation of several commercial dermal fillers from a selection thereof, to determine which filler will successfully reduce or eliminate the appearance of fine line wrinkles in the tear trough without causing undesirable discoloration due to Tyndall effect.

The physician introduces a first hyaluronic-acid based dermal filler intradermally behind the ear of the patient using linear threading technique. The physician applies oxymetazoline, a topical vasoconstricting agent, to the treated area.

The physician selects a second hyaluronic-acid based dermal filler from the selection. The physician introduces the second intradermally also behind the ear of the patient using linear threading technique, the second dermal filler being introduced about 1 cm apart from the site of the first dermal filler. The physician applies oxymetazoline, a topical vasoconstricting agent, to the treated area.

The physician instructs the patient to return the office the next day for follow up evaluation and to apply a cold compress to the treated area in the meantime.

The next day, the physician evaluates the appearance of the first and second dermal filler treated skin regions, and compares them to each other and to the untreated skin. The physician visually observes that the first dermal filler treatment area does not appear to show any discoloration relative to the untreated skin region, but the second dermal filler treatment area shows a slight blue discoloration. The physician further confirms these observations using a hand-held portable spectrophotometer and finds that % blue light reflected form the second filler treatment area is 38% compared to 35% for the first filler and 35.2% for untreated skin region.

Based on the measurements, the physician determines that the first dermal filler is the best selection for treating the fine line wrinkles in the patient.

### EXAMPLE 7

### Selecting a Suitable Dermal Filler for a Patient from a Selection of Dermal Fillers

A patient desires to have dermal filler treatment for treating age spots and skin hyper-pigmentation. A physician suggests using a new hyaluronic-acid based dermal filler formulation that contains vitamin C and vitamin A for treating skin pigmentation. The physician also suggests closely monitoring the treatment progress over several periodic evaluation of skin's color tone. The physician introduces the filler intradermally using depot technique, injecting the entire pigmented skin region. In three separate visits 2 weeks apart, the patient's treated skin region is evaluated by the physician using a hand-help portable spectrophotometer. During each visit, the physician scans the treated skin region as well as an adjoining untreated and non-pigmented skin region. For each measurement % blue, % Red, % Green, and total reflected light form skin is computed. After each bi-weekly skin assessment, the physician retreats the skin with the filler. At the final visit, the physician compares the spectral parameters (% blue, % Red, % Green, and total reflected light form skin) between the treated and untreated non-pigmented skin regions and finds that the parameters closely match up (refer to Table 1 below). The physician concludes that the therapy was successful.

Table 1 shows changes in % Red, % Green, % Blue and total reflected light from a pigmented skin (with and without treatment) and a non-pigmented untreated skin region from the same patient.

TABLE 1

| | Treated Skin Region | | | | Untreated |
|---|---|---|---|---|---|
| | (Pigmented Skin) | | | | Skin |
| | Before | After 1st | After 2nd | After 3rd | Region |
| | Treatment | treatment | treatment | treatment | (Non- |
| | (0 weeks) | (2 weeks) | (4 weeks) | (6 weeks) | pigmented) |
| % Red Color | 43 | 37 | 34 | 33 | 32 |
| % Green Color | 46 | 36 | 35 | 33 | 35 |
| % Blue Color | 11 | 27 | 31 | 34 | 33 |
| Total reflected | 30 | 50 | 55 | 61 | 60 |
| Light | | | | | |
| (scale 1-100) | | | | | |

### EXAMPLE 8

### Selecting a Suitable Dermal Filler for a Patient from a Selection of Dermal Fillers

A patient desires to have dermal filler treatment for treating skin dryness. A physician suggests using a new hyaluronic-acid based dermal filler formulation that contains vitamin B for treating skin hydration. The physician also suggests closely monitoring the treatment progress over several periodic evaluation of skin's hydration. The physician introduces the filler intradermally using depot technique, injecting the entire mid-face skin region. In three separate visits 2 weeks apart, the patient's treated skin region is evaluated by the physician using a hand-help portable spectrophotometer. During each visit, the physician scans the treated skin region. For each measurement total reflected light from skin is computed. After each bi-weekly skin assessment, the physician retreats the skin with the filler. At the final visit, the physician compares the change in total reflected light from treated skin to its initial value before the start of therapy. Physician finds that after treatment the skin has regained its hydration because the total light reflectance from treated skin has returned to normal healthy skin value. The physician concludes that the therapy was successful.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular method, technique or example described herein. As such, those skilled in the art could make numerous and various modifications or changes to or alternative configurations of the disclosed subject matter can be made in accordance with the teachings herein without departing from the spirit of the present specification. Changes in detail may be made without departing from the spirit of the invention as defined in the appended claims.

Groupings of alternative elements or embodiments of the invention disclosed herein are not to be construed as limitations. Each group member may be referred to and claimed individually or in any combination with other members of the group or other elements found herein.

What is claimed is:

1. A method for selecting a dermal filler composition comprising:
quantifying a color component in a skin region of a patient; and
selecting a dermal filler composition from a selection of different dermal filler compositions based on the quantified color component of the skin region of the patient.

2. The method of claim 1 wherein the step of quantifying comprises
introducing into a skin region a dermal filler composition to be evaluated; and
observing discoloration in the skin region.

3. The method of claim 2 wherein the step of observing comprises measuring at least one color component of the skin region.

4. The method of claim 3 wherein the color component measured includes a blue color component.

5. The method of claim 2 wherein the step of quantifying further comprising reducing blood flow to the skin region prior to or during the step of observing.

6. The method of claim 5 wherein the step of reducing blood flow comprises introducing a vasoconstrictor to the skin region.

7. The method of claim 5 wherein the step of reducing blood flow comprises applying a vasoconstricting agent to the skin region.

8. The method of claim 7 wherein the vasoconstricting agent is applied topically to the skin region.

9. The method of claim 2 wherein the observing comprises comparing a color component observed in the skin region with the same color component observed in an untreated skin region.

10. The method of claim 2 wherein the observing comprises measuring a color component in the skin region and comparing the color component with the same color component of an untreated skin region of the same patient.

11. The method of claim 3 wherein the measuring comprises assessing the discoloration using an electromechanical device.

12. The method of claim 3 wherein the measuring comprises assessing the discoloration using reflectance spectroscopy.

13. The method of claim 3 wherein the measuring comprises quantifying a blue color component of the skin region.

14. The method of claim 3 wherein the measuring comprises quantifying a percentage (%) of blue light reflected from the skin region.

15. The method of claim 3 wherein the measuring comprises quantifying a blue color component of the skin region using an electromechanical device.

16. The method of claim 3 wherein the measuring comprises quantifying a blue color component of the skin region using a spectrophotometer.

17. The method of claim 1 wherein the dermal filler is a hyaluronic acid-based dermal filler.

18. The method of claim 2 wherein the dermal filler is a substantially optically transparent, hyaluronic acid-based dermal filler and the skin region is observed for discoloration due to Tyndall effect.

19. A method for selecting a dermal filler composition from a plurality of different dermal filler compositions, the method comprising:
introducing into a skin region a dermal filler composition to be evaluated;
measuring a color component in the skin region relative to an untreated skin region of the patient; and
selecting a dermal filler composition based on the measured color component of the skin region of the patient.
